(19) United States
(12) Patent Application Publication
(10) Pub. No.: US 2024/0368107 A1
(43) Pub. Date: Nov. 7, 2024

(54) PREPARATION PROCESS OF QUERCETIN AND QUERCETIN THUS OBTAINED

(71) Applicant: HEALTHTECH BIO ACTIVES, S.L.U., BARCELONA (ES)

(72) Inventors: MARTA FERNÁNDEZ SUÁREZ, BENIEL (ES); FRANCISCO JAVIER LÓPEZ CREMADES, BENIEL (ES)

(21) Appl. No.: 18/654,212

(22) Filed: May 3, 2024

(30) Foreign Application Priority Data
May 5, 2023 (EP) 23382422.6

(51) Int. Cl. C07D 311/32

(52) U.S. Cl. CPC C07D 311/32

(57) ABSTRACT

A preparation process of quercetin of formula (1) comprising a) hydrolysis of rutin of formula (II) or an aqueous rutin containing extract, in the presence of water as only solvent and an acid in catalytic amounts; and b) isolation of the quercetin thus obtained. The quercetin thus obtained.

This application claims the benefit of European Patent Application EP23382422.6 filed on May 5, 2023.

### TECHNICAL FIELD

This invention relates to a preparation process of quercetin without using organic solvents. It also relates to the quercetin thus obtained.

### BACKGROUND ART

Quercetin is a natural flavonoid found abundantly in vegetables and fruits. Upon absorption in the small intestine, quercetin is metabolized immediately by enzymes in the epithelial cells and further metabolized by the liver. There is growing evidence suggesting that quercetin has therapeutic potential for the prevention and treatment of different diseases, including cardiovascular disease, cancer, and neurodegenerative disease. Mechanistically, quercetin has been shown to exert antioxidant, anti-inflammatory, and anticancer activities in a number of cellular and animal models, as well as in humans through modulating the signaling pathways and gene expression involved in these processes. The effects of quercetin concentration are varied with low doses (0-10 μM) resulting in chemoprevention, mid ranges (10-200 μM) resulting in mixed effects, and higher concentration (>200 μM) in pro-oxidant or potential direct therapeutic properties. From the studies presented, these lower concentrations appear to be achievable by diet, while the therapeutic concentrations might require supplementation or intravenous administration and result in little or no side effects.

Quercetin may be obtained from rutin which is a natural flavonoid. One possible source of rutin is found in Dimorphandra gardneriana, popularly known as Fava D'anta. But rutin is also found in other plant sources such as Sophora japonica or Uncaria elata among others.

Several methods are known for the extraction of rutin from natural sources. One commonly used method is the extraction with organic solvents. Extraction of rutin by heating with use of methanol as solvent is the main form known for its extraction. However, methanol is a toxic substance that can cause serious damage to human health, has peculiar narcotic properties, and causes irritation of the mucous membranes.

For example, D. O. Duarte et al., in Brazilian Journal of development 2020, vol. 6, pp. 16802-16818 describes a process for extracting rutin from fava D'anta using different organic solvents and concluded that methanol, the main solvent used in the extraction, can be replaced with ethanol or ethanol/water without compromising the rutin yield or purification degree. However, water extraction compromised the final rutin yield, this being around 8.3%, due to poor solubility of the substance in said solvent, even under high temperature. Another problem is that the process disclosed in this document requires several purification steps. It is necessary to purify rutin after its extraction, through crystallization using methanol and active charcoal, followed by filtration through a celite pad. Then, quercetin, which has been obtained by hydrolysis of the rutin using aqueous sulfuric acid (5%) and reflux for 6 h at 80° C., requires a purification through a crystallization process using ethanol and active charcoal.

On the other hand, CN104387357 describes the preparation of quercetin by acidic hydrolysis of rutin in spite of using enzymatic hydrolysis. The authors explain that enzymatic processes are complicated, the impurities are difficult to control, and the water after fermentation is difficult to treat. Therefore, the cost is high due to impurity removal processes and sewage treatment and other methods would be required. So, the authors propose the option of obtaining quercetin from rutin, using 2.5-5% w/w of sulfuric acid. However, still a purification step using methanol is needed to obtain a somewhat pure quercetin.

Hence, the prior art processes have the problem that they do not avoid organic solvents and are not feasible to provide a solvent free rutin or quercetin, meaning that they still contain an organic solvent in considerable amounts. Another problem is that the prior art conditions cause degradation of rutin and quercetin, which affects purity and, therefore, requires mandatory purification steps using organic solvents in order to provide an acceptable product. This makes the prior art processes more cumbersome and more expensive and eventually lower the yield because product will be lost during said purification steps. Moreover, since the prior art quercetin has the drawback of containing residual solvent due to the use of solvents in its preparation process and purification steps, it is not appropriate for the subsequent use in pharmacy or food industry.

Accordingly, from what is known in the art, it is derived that there is still the need of providing a preparation process of quercetin that allows obtaining the product with high yield, low content of impurities and no presence of residual solvents. There is a further need to provide a solvent free process for obtaining rutin and/or quercetin that is devoid of using any organic solvent, not even for purification purposes. Another need is the provision of such processes at industrial level to allow for large scale manufacturing of rutin and/or quercetin.

### SUMMARY OF INVENTION

Inventors have managed to extract and then hydrolyze subsequently rutin effectively in a solvent free or solvent-less fashion. In particular, the hydrolysis has been achieved with a much lower amount of acid, being a significant and crucial difference with respect to the known processes because it results in almost no degradation and allows directly obtaining quercetin with an at least 90% purity without the need for further purification steps. This process step is conducted in water and a catalytic amount of acid, yielding a product with a low content of impurities, without the need of a purification step of the quercetin, and in the absence of using organic solvents such as methanol or ethanol. Since no organic solvents have been used, the final product is especially advantageous for human consumption.

Furthermore, the process is more economical since the above-mentioned extra purifications do not have to be carried out, while at the same time higher yields are obtained. Moreover, it is more eco-friendly and avoids safety risks, since it does not use any toxic or otherwise hazardous organic solvents, and also due to the reduction of acids or their salts in the hydrolysis mother liqueurs, which makes the work-up simpler. Additionally, the process is feasible at large scale and thus viable for industrial purposes.

Thus, an aspect of the present invention relates to a preparation process of quercetin of formula (I) with a purity equal to or higher than 90% by weight, comprising: a) hydrolysis of rutin of formula (II) or an aqueous rutin containing extract in the presence of water as only solvent and an acid in a catalytic amount, at a temperature in a range from 100° C. to 150° C.; and b) isolation of the quercetin thus obtained;

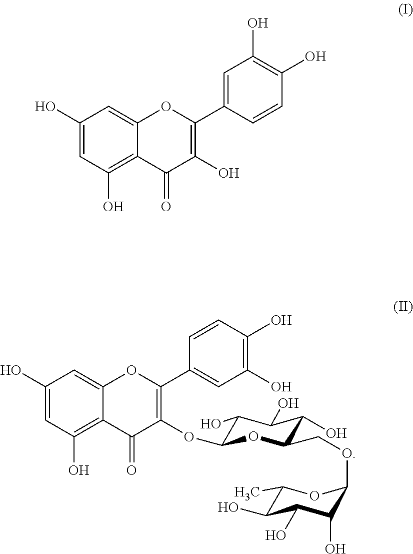

Another aspect of the present invention relates to quercetin with a purity equal to or higher than 90% and which is free of organic solvents, obtainable by the process as defined above. The quercetin obtained by the process of the present invention is also named solvent free quercetin.

### DETAILED DESCRIPTION OF THE INVENTION

### Definitions

All terms as used herein in this application, unless otherwise stated, shall be understood in their ordinary meaning as known in the art. Other more specific definitions for certain terms as used in the present application are as set forth below and are intended to apply throughout the description and claims.

Unless otherwise stated, all percentages mentioned herein are expressed in weight with respect to the total weight of the composition, provided that the sum of the amounts of the components is equal to 100%.

The term “comprised between” or “comprised in a range from x to y” as used herein refers to a range of values including the end points of the range. Any range of values disclosed herein regardless of the wording used, are encompassing the end points of the range in its scope.

The word “comprise” for the purposes of the present invention encompasses the case of “consisting of”.

The term “about” or “around” or “approximately” as used herein refers to a range of values ±10% of a specified value. For example, the expression “about 10” or “around 10” includes ±10% of 10, i.e., from 9 to 11.

The expression “catalytic amount of acid” as used herein refers to an amount of acid (a smaller amount than the stoichiometric amount) that is not consumed in the reaction but facilitates the hydrolysis reaction. The exact amount of acid needed to catalyze the hydrolysis can vary depending on the specific hydrolysis conditions, and the particular acid being used.

The term “aqueous rutin containing extract” as used herein refers to an extract of rutin from a natural source wherein the extraction has been conducted with water and, therefore, is free of organic solvents.

The term “solvent free rutin” refers to rutin which is free of organic solvents.

The term “solvent free quercetin” refers to quercetin which is free of organic solvents.

It is considered part of the invention a preparation process for obtaining quercetin, comprising the step of hydrolysing rutin of formula (II) or a rutin containing extract in the presence of water and a catalytic amount of acid, at a temperature in a range from 100° C. to 150° C. It is considered also part of the invention, conducting a previous step comprising extracting rutin from natural sources with water only, at a temperature in the range of 100° C. to 150° C. Finally, the aqueous preparation process for obtaining quercetin, as defined above, comprising the aqueous extraction of rutin from natural sources and/or the aqueous acidic hydrolysis of rutin or rutin containing extract, which is devoid of the use of organic solvents in any step and work-up, is also part of the invention.

Accordingly a preparation process of quercetin of formula (I) with a purity equal to or higher than 90% by weight according to the present invention may comprises the following steps: a) hydrolysis of rutin of formula (II) or an aqueous rutin containing extract, in the presence of water as only solvent and an acid in a catalytic amount, at a temperature in a range from 100° C. to 150° C.; and b) isolation of the quercetin thus obtained.

The hydrolysis step according to the present invention not only is carried out without the need of any organic solvent but also allows to obtain quercetin in excellent purity without requiring any specific purification method such as recrystallisation in methanol or ethanol as part of the work-up procedure.

The purity can be measured by HPLC The specific conditions are included in the experimental section. In a particular embodiment, the purity of the quercetin obtained by the process of the present invention is from 90 to 99% by weight. In another particular embodiment, the purity of the quercetin obtained by the process of the present invention is equal to or higher than 95% by weight, in particular from 95 to 99% by weight. In another particular embodiment, the purity of the quercetin obtained by the process of the present invention is equal to or more than 90%, 91%, 92%, 93%, 94%, 95%, 96%, 97%, 98% or 99%, all expressed as % by weight.

In a particular embodiment, the preparation process of quercetin of formula (I) with a purity equal to or higher than 90% by weight, consist of: a) hydrolysis of rutin of formula (II) or an aqueous rutin containing extract in the presence of water and an acid in a catalytic amount, at a temperature in a range from 100° C. to 150° C.; and c) isolation of the quercetin thus obtained.

The hydrolysis in the conditions tested in the present invention has been proved to be faster than in the conditions of the prior art, and additionally achieves better yields.

In a particular embodiment, in combination with any of the embodiments above or below, the process according to the present invention is that where the acid is selected from the group consisting of an inorganic acid and an organic acid, the inorganic acid is selected from the group consisting of hydrochloric acid, nitric acid, sulfuric acid, hydrobromic acid, hydroiodic acid, hydrofluoric acid, phosphoric acid, perchloric acid, boric acid, periodic acid, and sulfamic acid, and the organic acid is selected from the group consisting of, lactic acid, malic acid, succinic acid, citric acid, oxalic acid, tartaric acid, maleic acid, and malonic acid. In another particular embodiment, the acid is selected from the group of inorganic acids consisting of hydrochloric acid, nitric acid, sulfuric acid, hydrobromic acid, hydroiodic acid, phosphoric acid, perchloric acid. In a more particular embodiment, the acid is selected from the group of inorganic acids consisting of hydrochloric acid, nitric acid, and sulfuric acid. Particularly, the acid is sulfuric acid.

In a particular embodiment, in combination with any of the embodiments above or below, the catalytic amount of acid is in a range from 200 to 8000 ppm with respect to the mixture of step b). It has been found that higher amounts of acid should not be used because then degradation occurs in higher rates, the yield is reduced, and an additional purification step is required. In another particular embodiment, in combination with any of the embodiments above or below, the catalytic amount is in a range from 200 to 7000 ppm with respect to the mixture of step b). In another particular embodiment, catalytic amount is in a range from 300 to 7000 ppm with respect to the mixture of step b). In another particular embodiment of the preparation process, the catalytic amount is in a range from 400 to 6000 ppm with respect to the mixture of step b). In another particular embodiment of the preparation process, the catalytic amount is in a range from 500 to 5000 ppm with respect to the mixture of step b). In another particular embodiment of the preparation process, the catalytic amount is in a range from 600 to 4000 ppm with respect to the mixture of step b). In another particular embodiment of the preparation process, the catalytic amount is in a range from 700 ppm to 3000 ppm with respect to the mixture of step b). In another particular embodiment of the preparation process, the catalytic amount is in a range from 800 to 2000 ppm with respect to the mixture of step b). In another particular embodiment of the preparation process, the catalytic amount is in a range from 800 to 1800 ppm with respect to the mixture of step b). In another particular embodiment of the preparation process, the catalytic amount is in a range from 1000 to 1700 ppm with respect to the mixture of step b). In another particular embodiment of the preparation process, the catalytic amount is in a range from 1200 to 1600 ppm with respect to the mixture of step b). More in particular, 800, 900, 1000, 1100, 1200, 1300, 1400, 1500, 1600, 1700, 1800, 1900, 2000 ppm or any combination thereof. The acids used are water soluble and do not require special treatment to separate them from quercetin.

In another particular embodiment of the preparation process, in combination with any of the embodiments above or below, the rutin is in a concentration in the mixture in a range from 3 to 15% weight/volume. In another particular embodiment of the preparation process, the rutin is in a concentration in the mixture in a range from 4 to 15% weight/volume. In another particular embodiment of the preparation process, the rutin is in a concentration in the mixture in a range from 4 to 13% weight/volume. In another particular embodiment of the preparation process, the rutin is in a concentration in the mixture in a range from 5 to 12% weight/volume. In another particular embodiment of the preparation process, the rutin is in a concentration in the mixture in a range from 5 to 10% weight/volume. In another particular embodiment, the rutin is in a concentration in the mixture in a range from 5, 6, 7, 8, 9, or 10% weight/volume, or any combination thereof. It has been found that at too low concentrations, the risk of degradation is higher, while when the concentration is too high, there may be insufficient dissolution that also affects negatively the outcome of the process.

In another particular embodiment of the preparation process, in combination with any of the embodiments above or below, the hydrolysis is conducted at a temperature of 100-150° C. In another particular embodiment of the preparation process, the hydrolysis is conducted at a temperature of 105-140° C. In another particular embodiment of the preparation process, the hydrolysis is conducted at a temperature of 105-130° C. In another particular embodiment of the preparation process, the hydrolysis is conducted at a temperature of 105-125° C. In another particular embodiment of the preparation process, the hydrolysis is conducted at a temperature of 105, 106, 107, 108, 109, 110, 111, 112, 113, 114, 115, 116, 117, 118, 119, 120, 121, 122, 123, 124, or 125° C. At these temperatures, rutin may be completely dissolved, for instance, at a concentration of 5-10% weight/volume which allows a more efficient hydrolysis be conducted and avoid the formation of Impurities. It has been found that at lower temperatures, no full rutin dissolution may be obtained affecting negatively the outcome of the process, while at higher temperatures degradation occurs resulting in more impurities and requiring then additional purification steps.

In another particular embodiment of the preparation process, in combination with any of the embodiments above or below, the hydrolysis is carried out during a period of at least 2 hours. In another particular embodiment of the preparation process, the hydrolysis is carried out during a period of from 2 hours to 6 hours. In another particular embodiment of the preparation process, the hydrolysis is carried out during a period of from 2 hours to 5 hours. In another particular embodiment, the hydrolysis is carried out during a period of 2, 3, 4, 5 hours or any combination thereof.

In another particular embodiment of the preparation process, in combination with any of the embodiments above or below, the isolation of quercetin comprises cooling the media at a temperature in a range from 60 to 98° C., collecting the product, e.g., by filtering the product, and optionally washing it with water. In another particular embodiment of the preparation process, the isolation of quercetin comprises cooling the media at a temperature in a range from 80 to 95° C., collecting the product, e.g., by filtering the product, and optionally washing it with water. The quercetin thus obtained may be dried until constant weight at an appropriate temperature, for instance at a temperature in the range from 40 to 70° C., in particular, at 65° C. In another particular embodiment of the preparation process, the isolation of quercetin is not followed by any purification method that requires organic solvents.

The rutin starting material of the hydrolysis may be commercial rutin or may be a rutin containing extract obtained from natural sources.

In the case of commercial rutin, it should have a purity of at least 65% by weight. The rutin extract may be obtained from any natural source that contains rutin. Preferably, the natural source contains rutin in such amounts that makes its extraction commercially viable. However, it is also contemplated to use genetically modified natural sources that have an enriched rutin content compared with their non-genetically modified variants. The amount of rutin in the natural sources can vary depending on factors such as growing conditions and extraction conditions. Thus, the purity and yield may vary depending on the quality of the starting product (variation according to region, harvest date, and others, as always happens with vegetable raw materials).

The rutin extract used as starting material of the hydrolysis may be an extract with a content of rutin equal to or higher than 65% by weight. In particular, the rutin extract may have a content of rutin equal to or higher than 75% by weight, more in particular, a content of rutin equal to or higher than 75% by weight, even more particular, a content of rutin equal to or higher than 79% by weight, still even more particular, a content of rutin equal to or higher than 80% by weight. In another particular embodiment, the rutin starting material either commercial rutin or a rutin extract according to the present invention has a purity in a percentage in a range from 70 to 85% of Rutin by weight.

The crude rutin obtained from the extraction process of the invention described in detail below contains mainly rutin and as minor impurity isoquercetin. Isoquercetin may be also converted into quercetin during the hydrolysis step of the invention. Generally, the crude rutin of the rutin extract may contain around 65-85% by weight of rutin and a content of isoquercetin equal to or higher than 10% by weight, in particular, equal to or higher than 15% by weight. Generally, the amount of isoquercetin, if present, varies and it is in an amount up to 20% by weight, preferably and commonly in the range between 10 to 20% by weight, more preferably in the range between 13 to 18% by weight.

In a particular embodiment of the preparation process of the present invention, in combination with any of the embodiments above or below, the rutin containing natural source is selected from the group consisting of Fava D'anta, Sophora japonica, Uncaria elata, buckwheat, onions, capers, dill, coriander, radish, viola tricolor, Morus alba, and white mulberry. In another particular embodiment of the preparation process of the present invention, the rutin containing natural source is selected from the group consisting of fava D'anta, Sophora japonica, and buckwheat. In another particular embodiment of the preparation process of the present invention, the rutin containing natural source is a fava D'anta, and Sophora japonica. In another particular embodiment of the preparation process of the present invention, the rutin containing natural source in the extraction step is a fava D'anta.

The preparation process of the present invention may comprise extracting rutin of formula (II) from a rutin-containing natural source which may be a vegetable or plant source as defined above, where the extraction process comprises: 1) mixing a grinded rutin-containing natural source in water at a temperature in a range from 100° C. to 150° C.; 2) cooling the mixture of step 1) to a first temperature being lower than the temperature of step 1) and separating the residual vegetal material from the rutin-containing extract; and 3) optionally, isolating the product thus obtained.

In a particular embodiment, the first temperature of step 2) is in a range of 60 to 97° C.

Advantageously, the extraction process of the present invention is conducted in the absence of any organic solvent. In a particular embodiment, the extraction process allows obtaining a crude rutin extract that does not require any further purification step with organic solvents and can thus be directly used in the subsequent hydrolysis for obtaining a quercetin free of organic solvents, in particular, the crude rutin extract is a solvent free rutin extract and also in particular, the quercetin obtained is a solvent free quercetin.

The product obtained from the extraction process is a product rich in rutin such as a mixture of rutin and isoquercetin described above. As a way of example, it may be a mixture of 78-82% by weight of rutin and 14-16% by weight of isoquercetin or any other crude rutin extract in any of the proportions mentioned above.

Advantageously, the extraction process of the present invention gives rise to higher yields than the known processes disclosed in the prior art. The process of the present invention further comprises the hydrolysis of the rutin extract thus obtained in the conditions disclosed above.

In a particular embodiment, in combination with any of the embodiments above or below, the preparation process of the present invention is that which consist of the following steps: (A) extracting rutin of formula (II) from a rutin-containing natural source which is a vegetable or plant source, selected from the group consisting of Fava D'anta, Sophora japonica, and Uncaria elata, buckwheat, onions, capers, dill, coriander, radish, viola tricolor, Morus alba, and white mulberry, where the extraction process consists of: 1) mixing the grinded rutin-containing natural source in water at a temperature in a range from 100° C. and 150° C.; 2) cooling the mixture of step 1) to a first temperature being lower than the temperature of step 1) and separating the residual vegetal material from the rutin-containing extract; 3) optionally, isolating the product thus obtained; and (B) hydrolysis of the crude rutin extract thus obtained in the conditions disclosed above.

In a particular embodiment, the first temperature of step 2) is in a range of 60 to 97° C.

In a particular embodiment, in combination with any of the embodiments above or below, the process comprises grinding the rutin containing natural source.

In a particular embodiment of the preparation process of the present invention, in combination with any of the embodiments above or below, in step 1) of the extraction step the temperature is in the range from 100° C. to 140° C. In another particular embodiment of the preparation process of the present invention, in step 1) of the extraction step the temperature is 105-140° C. In another particular embodiment of the preparation process of the present invention, in step 1) of the extraction step the temperature is 105-130° C. In another particular embodiment of the preparation process of the present invention, in step 1) of the extraction step the temperature is 105, 106, 107, 108, 109, 110, 111, 112, 113, 114, 115, 116, 117, 118, 119, 120, 121, 122, 123, 124, 125, 126, 127, 128, 129, 130° C., or any combination thereof. At these working temperatures, it has been found that the extraction is very efficient, and the formation of impurities is avoided.

In another embodiment of the preparation process of the present invention, in combination with any of the embodiments above or below, in step 1) of the extraction step, the mixing step is carried out during a period of at least 15 minutes, in particular in a range from 15 minutes up to 5 hours.

In another embodiment of the preparation process of the present invention, in combination with any of the embodiments above or below, in the extraction step, the concentration of rutin natural source in water is in the range from 5 to 15% w/v. In another embodiment of the preparation process of the present invention, in the extraction step, the concentration of rutin natural source in water is in the range from 5 to 12% w/v. In another embodiment of the preparation process of the present invention, in the extraction step, the concentration of rutin natural source in water is 5, 6, 7, 8, 9, 10, 11, 12% w/v or any combination thereof.

In another particular embodiment of the preparation process of the present invention, in combination with any of the embodiments above or below, the cooling step 2) of the extraction process comprises cooling to a first temperature being lower than the temperature of step 1) of the extraction, wherein the first temperature is in a range from 65 to 95° C. At this first temperature of cooling step 2), the vegetal residues are separated from the rutin-containing extract.

The rutin-containing extract obtained after separating the vegetal residues can be used either directly for the hydrolysis step or can be cooled to a second temperature being lower than the first temperature. In a particular embodiment, the rutin-containing extract can be cooled to a second temperature, ranging from 20 to 85° C. Even at this second temperature, the rutin-containing extract can optionally be either used directly in the hydrolysis step of the process, i.e., without the isolation step 3) of the extraction process, or can be used to isolate in step 3) the product, i.e., rutin, thus obtained. In another particular embodiment, the process comprises cooling to a second temperature ranging from 40 to 85° C. and either use the rutin-containing extract at the second temperature directly for the hydrolysis process or isolate the product thus obtained in step 3 of the extraction process. The mixture can be maintained at the chosen second temperature to favor the crystallization of crude rutin for the necessary period of time and then isolate the rutin by, for example, filtering.

In another particular embodiment of the preparation process of the present invention, in combination with any of the embodiments above or below, the isolation step 3) comprises collecting the product at the second temperature and washing it with water. The product may be collected for instance by filtration.

Generally, the crude rutin extract thus obtained is used as such in the preparation process of quercetin from rutin, i.e., without isolating the rutin thus obtained. If desired, the crude rutin extract may be dried in an oven at an appropriate temperature such as for example 40-70° C., and in particular around 65° C.

The extraction of rutin by the process described above provides a crude rutin extract with a purity generally, between 65-85% weight. Generally, the yield is between 15-36% by weight.

After hydrolysis according to the invention the quercetin product obtained has a purity equal to or higher than 90% by weight, generally the yield of the hydrolysis step is 45-51% by weight.

The global yield is comprised between 7-18% by weight. “Native extract ratio” as used herein indicates how much material has been extracted from the vegetable source. The native extract ratio varies depending on the quality of the natural source. In a particular embodiment the natural source is Fava D'anta, and the native extract ratio Fava D'anta: quercetin varies from 5:1 to 11:1 by weight; meaning that from 5-11 g of Fava D'anta, 1 g of quercetin can be obtained depending on the quality of the Fava D'anta.

As quercetin obtained directly by the process of the present invention has already a high purity, there is no need to further purify it, which is a very significant advantage of the process of the present invention. Thus, as mentioned above the process of obtaining quercetin from rutin of the present invention does not require any purification step, in particular it does not need any purification step using organic solvents, nor for the extracted rutin obtained in the extraction step nor for the quercetin obtained after hydrolysis and, thus, allows obtaining quercetin directly with a purity equal to or higher than 90% in weight, even equal to or higher than 95% by weight.

Accordingly, in a particular embodiment of the preparation process of the present invention, in combination with any of the embodiments above or below, the process comprises a first the step of extracting the rutin from a natural source and then the step of hydrolysis, both according to the present invention, and wherein the process is conducted without carrying out a further purification step of the rutin extract. In another particular embodiment of the preparation process of the present invention, in combination with any of the embodiments above or below, the process comprises a first the step of extracting the rutin from a natural source and then the step of hydrolysis, both according to the present invention, and wherein the process is conducted without carrying out a further purification step of the quercetin product. In another particular embodiment of the preparation process of the present invention, in combination with any of the embodiments above or below, the process comprises a first the step of extracting the rutin from a natural source and then the step of hydrolysis, both according to the present invention, and wherein the process is conducted without carrying out a further purification step neither of the rutin nor of the quercetin product. In particular, the process of the present invention is conducted without using any organic solvent, and in particular any purification step using organic solvents.

It is also part of the invention the quercetin with a degree of purity equal to or higher than 90% by weight, preferably, with a purity equal to or higher than 95% by weight, and which is free of organic solvent, obtainable by the process as defined above, including any of the particular embodiments disclosed above, the process comprising the steps of extraction of rutin and subsequent hydrolysis as described above. As far as the inventors know, all the processes of the prior art employ organic solvents in at least one of the steps for obtaining quercetin, in particular they need to conduct recrystallization with organic solvents such as methanol and ethanol in order to purify the quercetin, which implies that inevitably certain amounts of residual solvents are found in the product obtained from the prior art processes. In a particular embodiment, the quercetin obtainable by the process of the present invention is free of residual organic solvents because no organic solvents have been used throughout the whole process.

All the embodiments disclosed above for the process are also particular embodiments of the product herein disclosed.

The Quercetin obtained according to the present invention can be formulated into any compositions, such as pharmaceutical compositions, for example, drugs. It can also be used as a nutritional supplement or food additive.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

High performance liquid chromatography analysis was carried out with an Agilent Technologies 1290 Infinity II LC System, including a quaternary pump, thermostat, autosampler and UV detector. An Hypersil BDS C18 (250×4.6 mm, 5 μm) was used for separation. Mobile phase composition: methanol, water, and phosphoric acid (100:100:1); column temperature 35° C., Flow rate 1 L/min; UV detector wavelength 370 nm.

### Example 1—Extraction of Rutin from Fava D'Anta

35 g of Fava D'anta was mixed with 250 mL of water (14% w/v) at 115° C. for 2 hours. The mixture was cooled down to 90° C., and then filtrated to separate the solid vegetable residue. A liquid extract containing Rutin was obtained. The extract was cooled down to 25° C. with agitation overnight. The precipitated crude rutin was filtrated and washed with water and finally dried at 65° C. Yield: 4.66 g of Rutin (13% yield) with a purity of the crude product in rutin of 73% and in isoquercetin 13-15%.

### Example 2—Preparation of Quercetin

75 g of rutin of 74.8% purity and 12.9% of isoquercetin were dissolved with 500 mL H₂O (15% w/v) and 60 mL of H₂SO₄ 1% at 120° C. for 2 hours. Afterwards, the medium was cooled down to 90° C., and then the Quercetin was filtrated and washed with water.

Quercetin was dried under vacuum to obtain 30 g of quercetin (yield of 40%) with 90.9% purity.

### CITATION LIST

### Patent Literature

CN104387357

### Non-Patent Literature

D. O. Duarte et al., in Brazilian Journal of development 2020, vol. 6, pp. 16802-16818

1. A preparation process of quercetin of formula (I) with a purity equal to or higher than 90% by weight, 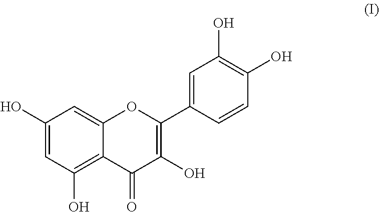 [SMILES: O=c1c(O)c(-c2ccc(O)c(O)c2)oc2cc(O)cc(O)c12]
comprising:
a) hydrolysis of rutin of formula (II) or an aqueous rutin containing extract, 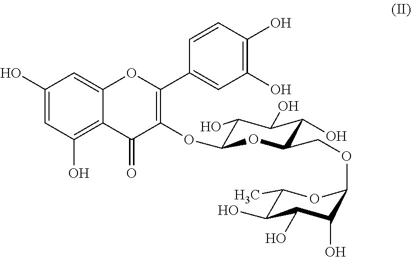
in the presence of water as only solvent and an acid in a catalytic amount, at a temperature in a range from 100° C. to 150° C.; and
b) isolation of the quercetin thus obtained.

2. The preparation process according to claim 1, wherein the catalytic amount is in a range from 200 to 8000 ppm with respect to the mixture of step a).

3. The preparation process according to claim 2, wherein the catalytic amount is in a range from 400 to 6000 ppm in the mixture of step a).

4. The preparation process according to claim 1, wherein the acid is selected from the group consisting of an inorganic acid and an organic acid,
the inorganic acid is selected from the group consisting of hydrochloric acid, nitric acid, sulfuric acid, hydrobromic acid, hydroiodic acid, hydrofluoric acid, phosphoric acid, perchloric acid, boric acid, periodic acid, and sulfamic acid, and
the organic acid is selected from the group consisting of lactic acid, malic acid, succinic acid, citric acid, oxalic acid, tartaric acid, maleic acid, and malonic acid.

5. The preparation process according to claim 2, wherein the acid is selected from the group consisting of an inorganic acid and an organic acid,
the inorganic acid is selected from the group consisting of hydrochloric acid, nitric acid, sulfuric acid, hydrobromic acid, hydroiodic acid, hydrofluoric acid, phosphoric acid, perchloric acid, boric acid, periodic acid, and sulfamic acid, and
the organic acid is selected from the group consisting of lactic acid, malic acid, succinic acid, citric acid, oxalic acid, tartaric acid, maleic acid, and malonic acid.

6. The preparation process according to claim 4, wherein the acid is selected from the group consisting of hydrochloric acid, nitric acid and sulfuric acid.

7. The preparation process according to claim 5, wherein the acid is selected from the group consisting of hydrochloric acid, nitric acid and sulfuric acid.

8. The preparation process according to claim 1, wherein the rutin is in a concentration in the mixture in a range from 5 to 15% weight/volume (w/v).

9. The preparation process according to claim 1, wherein the isolation of quercetin comprises cooling the media at a temperature in a range from 60 to 98° C., collecting the product, and optionally washing it with water.

10. The preparation process according to claim 1, wherein the aqueous rutin containing extract used as starting material for the hydrolysis has a content of rutin equal to or higher than 65% by weight.

11. The preparation process according to claim 1, wherein the process further comprises a previous step which comprises extracting rutin of formula (II) from a rutin containing natural source, 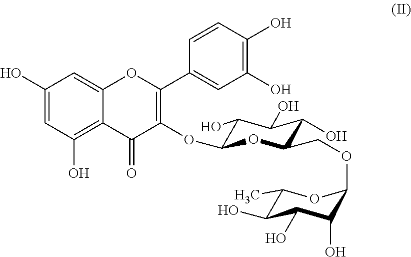
wherein the extraction process comprises:
1) mixing a grinded rutin natural source in water at a temperature in a range from 100° C. to 150° C.;
2) cooling the mixture of step 1) to a first temperature being lower than the temperature of step 1) and separating the residual vegetal material from the rutin-containing extract; and
3) optionally, isolating the product thus obtained.

12. The process according to claim 1, which is conducted in the absence of an organic solvent.

13. The preparation process according to claim 11, which is conducted in the absence of an organic solvent.

14. The preparation process according to claim 11, wherein the rutin containing natural sources is selected from the group consisting of Fava D'anta, Sophora japonica, Uncaria elata, buckwheat, onions, capers, dill, coriander, radish, viola tricolor, Morus alba, and white mulberry.

15. The preparation process according to claim 14, wherein the rutin source is selected from Fava D'anta, Sophora japonica, and buckwheat.

16. The preparation process according to claim 11 wherein the temperature in step 1) is in a range from 105 to 125° C.

17. The preparation process according to claim 11, wherein the concentration of rutin natural source in water is 5-15% (w/v).

18. Quercetin with a purity equal to or higher than 90% by weight and which is free of organic solvent, obtained by the process of claim 1.

19. Quercetin according to claim 18, wherein the catalytic amount of the acid is in a range from 200 to 8000 ppm with respect to the mixture of step a).

20. Quercetin according to claim 19, wherein the acid is selected from the group consisting of hydrochloric acid, nitric acid, and sulfuric acid.

21. Quercetin according to claim 18, wherein the process further comprises a previous step which comprises extracting rutin of formula (II) from a rutin containing natural source, 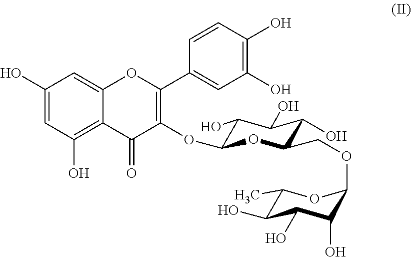
wherein the extraction process comprises:
1) mixing a grinded rutin natural source in water at a temperature in a range from 100° C. to 150° C.;
2) cooling the mixture of step 1) to a first temperature being lower than the temperature of step 1) and separating the residual vegetal material from the rutin-containing extract; and
3) optionally, isolating the product thus obtained.

22. Quercetin according to claim 21, wherein the natural source is selected from Fava D'anta, Sophora japonica, and buckwheat.
